(19) United States
(12) Patent Application Publication
(10) Pub. No.: US 2022/0238191 A1
(43) Pub. Date: Jul. 28, 2022

(54) MOLECULAR MODELING WITH MACHINE-LEARNED UNIVERSAL POTENTIAL FUNCTIONS

(71) Applicant: ACCUTAR BIOTECHNOLOGY INC., Brooklyn, NY (US)

(72) Inventors: Jie FAN, New York, NY (US); Ke LIU, Sunnyvale, CA (US); Xiangyan SUN, Shanghai (CN)

(73) Assignee: ACCUTAR BIOTECHNOLOGY INC., Brooklyn, NY (US)

(21) Appl. No.: 17/587,212

(22) Filed: Jan. 28, 2022

Related U.S. Application Data
(60) Provisional application No. 63/142,733, filed on Jan. 28, 2021.

(51) Int. Cl. G16C 20/70; G16C 20/30

(52) U.S. Cl. CPC G16C 20/70; G16C 20/30

(57) ABSTRACT

The present disclosure provides methods and apparatuses for molecular modeling with machine-learned universal potential functions. An exemplary method includes: determining a data structure representing chemical identities of atoms in a molecule; training an energy potential model using a training set comprising the data structure, true conformations of the molecule, and false conformations of the molecule; determining, using the trained energy potential model, a potential function associated with the molecule; or determining a conformation of the molecule based on potential function.

### CROSS-REFERENCE TO RELATED APPLICATIONS

The disclosure claims the benefits of priority to U.S. Provisional Application No. 63/142,733, filed on Feb. 28, 2021, which is incorporated herein by reference in its entirety.

### TECHNICAL FIELD

The present disclosure generally relates to the technical field of molecular modeling and, more particularly, to computational methods for molecular modeling with machine-learned universal potential functions.

### BACKGROUND

Molecular modeling started before the development of the modern digital computer, with one of the first simulations performed using wooden balls connected by springs in the basic ball-and-stick model. With the development of computers that are one million times faster, the basic representation of molecules has not changed much in modern molecular modeling.

The basic approach to molecular modeling is to define a force field for quantizing the forces between molecular atoms. A force field usually covers the bond, angle, and dihedral tension forces of chemical compounds. Most modern force fields share the same structure and parametrization process. Energy terms are usually defined as a result of a compromise between physical intuition and computational feasibility. Atoms are categorized into tens or sometimes hundreds of handpicked types. Parametrization is carried out by fitting the system to a specific dataset, usually higher-level computational results from a variety of molecules to ensure some desired properties such as co-planarity of aromatic rings, and plausible fit of bond length and angles. However, functions as complicated as atomic/molecular interactions may be beyond the expressiveness of quadratic and polynomial functions, or any other fixed combination of common mathematical functions. There are also many existing force field implementations. FIG. 1 shows examples of energy terms and atom types in the CHARMM General Force Field (CHARMMCGEN FF) (see, e.g., Kenno Vanommeslaeghe, E Prabhu Raman, and Alexander D MacKerell Jr. 2012. Automation of the CHARMM General Force Field (CGenFF) II: assignment of bonded parameters and partial atomic charges. Journal of chemical information and modeling 52, 12 (2012), 3155-3168) and the universal force field (UFF) (see, e.g., Anthony K Rappé, Carla J Casewit, KS Colwell, William A Goddard III, and W Mason Skiff 1992. UFF, a full periodic table force field for molecular mechanics and molecular dynamics simulations. Journal of the American chemical society 114, 25 (1992), 10024-10035).

The major drawback of existing force fields is that the atom types, energy term functions, and parameters are usually hand fitted on small datasets. This makes the energy fields less generalized and usually only works well for the specifically tuned field. Correction terms usually need to be added to force fields when working with other system or rare atom types, which complicates the system greatly. With the recent renaissance of neural networks and artificial intelligence came many attempts of using neural networks as a method of modeling molecular systems. Many of these attempts used neural networks to calculate a higher level of abstraction of molecules as a feature extraction tool to replace hand-picked fingerprints. The Graph Convolutional Network (GCN) is the preferred method to model molecular information as it allows a seamless and lossless way of representing the input molecule as a graph. GCN has been used to model physical and pharmaceutical properties of molecular compounds, as well as used for molecule generation.

For example, Schnet (see, e.g., Kristof T Schütt, Huziel E Sauceda, P-J Kindermans, Alexandre Tkatchenko, and K-R Müller. 2018. SchNet—A deep learning architecture for molecules and materials. The Journal of Chemical Physics 148, 24 (2018), 241722) is a machine learning framework for molecules, which encodes atom types using learned embeddings. This work showed such embeddings, when mapped by the first and second principal component, can be used to group atoms of the same group into the same cluster. This network was originally trained on the Quantum Machine 9 (QM9) dataset, which consists only of hydrogen, carbon, nitrogen, oxygen and fluorine elements, but it is now trained on The Materials Project database that contains mainly inorganic compounds and 50,000 molecules. In the training process, each progression involves converting atom positions into absolute positions r to describe the local chemistry environment of an atom. Some tricks such as calculating pairwise distances instead of using relative positions are employed to implement rotational invariance.

The benefit of the neural network-based approach is that it can learn important features and interactions between atoms and bonds automatically. The main drawback is the lack of interpretation of the trained model, i.e., it is difficult to explain how the model works for a given input molecule. This makes the neural network a poor replacement for traditional force fields, as the latter have clear physical correspondence and are established on a variety of molecule modeling tasks.

The present disclosure provides methods and apparatuses for solving one or more problems described above.

### SUMMARY

In some embodiments, an exemplary method for predicting molecular conformation is provided. The method includes: determining a data structure representing chemical identities of atoms in a molecule; training an energy potential model using a training set comprising the data structure, true conformations of the molecule, and false conformations of the molecule; determining, using the trained energy potential model, a potential function associated with the molecule; or determining a conformation of the molecule based on potential function.

In some embodiments, an exemplary apparatus includes at least one memory for storing instructions and at least one processor. The at least one processor is configured to execute the instructions to cause the apparatus to perform: determining a data structure representing chemical identities of atoms in a molecule; training an energy potential model using a training set comprising the data structure, true conformations of the molecule, and false conformations of the molecule; determining, using the trained energy potential model, a potential function associated with the molecule; or determining a conformation of the molecule based on potential function.

In some embodiments, an exemplary non-transitory computer readable storage medium stores a set of instructions. The set of instructions are executable by one or more processing devices to cause an apparatus to perform: determining a data structure representing chemical identities of atoms in a molecule; training an energy potential model using a training set comprising the data structure, true conformations of the molecule, and false conformations of the molecule; determining, using the trained energy potential model, a potential function associated with the molecule; or determining a conformation of the molecule based on potential function.

### BRIEF DESCRIPTION OF THE DRAWINGS

The accompanying drawings, which are incorporated in and constitute a part of this specification, illustrate embodiments consistent with the present disclosure and, together with the description, serve to explain the principles of the present disclosure.

FIG. 1 shows energy terms and atom typing used in existing atomic force fields.

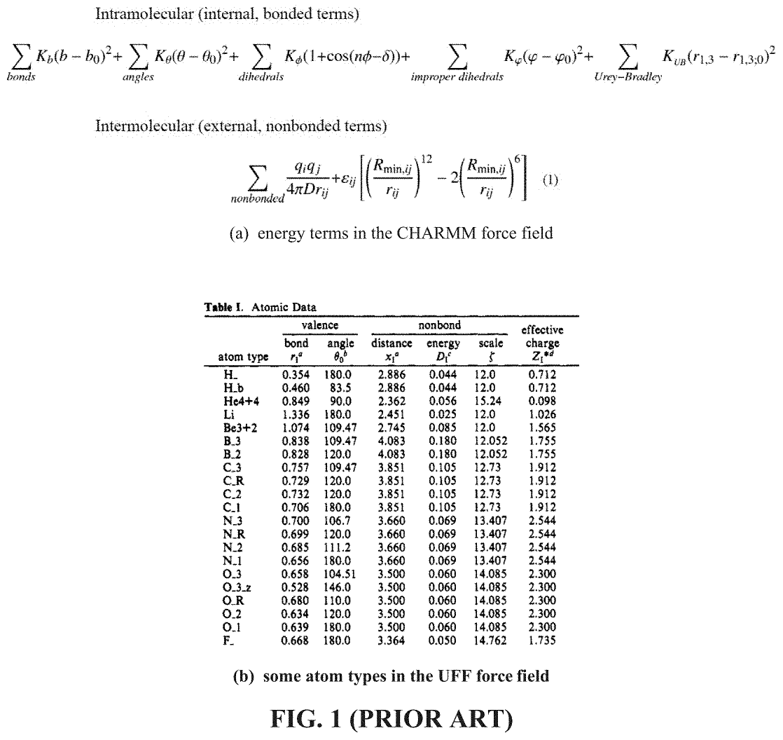

FIG. 2 is a schematic diagram illustrating exemplary potential terms used in the disclosed potential model, consistent with disclosed embodiments of the present disclosure.

FIG. 3 is a schematic diagram illustrating unbonded angle and dihedral in a hydrogen bond, consistent with disclosed embodiments of the present disclosure.

FIG. 4 shows an exemplary algorithm of gradient descent for molecular conformation optimization, consistent with disclosed embodiments of the present disclosure.

FIG. 5 shows an exemplary algorithm of negative sampling and modeling training, consistent with disclosed embodiments of the present disclosure.

FIG. 6 shows an exemplary algorithm of general conformation optimization, consistent with disclosed embodiments of the present disclosure.

FIG. 7 shows an exemplary algorithm of leave-one-out side chain prediction (LOO prediction), consistent with disclosed embodiments of the present disclosure.

FIG. 8 is a schematic diagram illustrating ligand preparation, consistent with disclosed embodiments of the present disclosure.

FIG. 9 shows an exemplary algorithm of ligand conformation generation for rigid components, consistent with disclosed embodiments of the present disclosure.

FIG. 10 is a schematic diagram illustrating anchor and grow process for ligand docking, consistent with disclosed embodiments of the present disclosure.

FIG. 11 shows an exemplary algorithm for performing the process in FIG. 10, consistent with disclosed embodiments of the present disclosure.

FIG. 12 shows an exemplary algorithm for small compound docking, consistent with disclosed embodiments of the present disclosure.

FIG. 13 is a block diagram of a device for performing the disclosed methods, consistent with disclosed embodiments of the present disclosure.

FIG. 14 is a flow chart of a neural network based method for predicting molecular conformation, consistent with disclosed embodiments of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the invention. Instead, they are merely examples of apparatuses and methods consistent with aspects related to the invention as recited in the appended claims. Particular aspects of the present disclosure are described in greater detail below. The terms and definitions provided herein control, if in conflict with terms and/or definitions incorporated by reference.

The present disclosure provides a hybrid framework combining the strength of both the force field and neural network-based approaches. Specifically, the disclosed methods use a neural network-based energy potential function which has the advantage of being trained on a large set of available crystal molecule structures, while keeping the benefits of traditional force fields as it is possible to do molecular dynamics, such as simulations, and it can also be applied to side chain prediction and docking tasks. By having a trained neural network model, the model can generally adapt to all types of chemical systems such as proteins and ligands. Using a fully automatic training process, our method eliminates the manual tuning steps involved in traditional force fields. The dynamic negative sampling and bootstrapping algorithm used makes the potential functions have good local minimums at ground truth conformations. As a result, the obtained neural network-based energy potential function shows superior performance on a variety of tasks over existing methods.

The proposed methods for generating machine-learned universal potential functions are described in detail below.

Consistent with the disclosed embodiments, a trained neural network is used to perform atom type embedding. Atom types are used to distinguish the chemical identities of different atoms. Atom types are chosen to satisfy that atoms with the same electric, chemical, and structural properties have the same atom type. Atom types are conventionally calculated using predefined rules, which resemble corresponding chemical properties. A simple example of such rule is to define each (element, hybridization) pair as a separated atom type.

In the present disclosure, an alternative known as learned atom type embedding is used. Instead of predefining rules of how to group or distinguish atoms, a neural network is trained to give embeddings of atom types. Such embeddings are then used in downstream models. In this way, the model can learn arbitrary complex atom type hierarchies, not limited to existing human chemical knowledge. The model also has the capability to encode more chemical information than just a single identification in such embedding. Different atom types may share some chemical properties, such as those with the same element number. Using an embedding-like distributed representation instead of hard defined types could also leverage such similarities.

The atom embedder uses the graph representation of a molecule, where the vertices correspond to atoms and edges corresponds to bonds. In addition, the following chemical relevant features are extracted and associated with the vertices (atoms) and edges (bonds).

The vertex/atom features include:
Element: the element type of the atom.
Charge: the electrostatic charge of the atom.
Radius: the van der Wells radius and covalent radius of the atom.
In ring: whether the atom is part of a ring.
In aromatic ring: whether the atom is part of an aromatic ring.

The edge/bond features include:
Bond type: the type of bond, one of {single, double, triple, aromatic}.
Same ring: whether the two atoms are in the same ring.

After an input molecule is transformed into a graph, the atom embeddings of each atom is calculated by a graph convolution-like model. For example, details of the graph convolution-like model are described in Ke Liu, Xiangyan Sun, Lei Jia, Jun Ma, Haoming Xing, Junqiu Wu, Hua Gao, Yax Sun, Florian Boulnois, and Jie Fan. 2019. Chemi-Net: a molecular graph convolutional network for accurate drug property prediction. International journal of molecular sciences 20, 14 (2019), 3389, which is incorporated in the present disclosure by reference. In each graph convolution layer, the embeddings of each atom Eᵢ is updated by information from neighboring atoms:

Eᵢ^{t}=Reduce ({{Eⱼ^{t-1}, B (i, j)}|j ∈ Neighbor(i)}) (Eq. 1)

where the initial embeddings Eᵢ⁰ is the predefined atom features, B(i,j) is the bond features. The Reduce(·) function is a set reduction as defined in section for reducing information from a set of embeddings into one.

Each of the graph convolution aggregates the information for each atom from one bond further in the molecule graph. After k steps the atom's embeddings contain information from k bonds away for each atom. The extracted atom embeddings are then fed into the downstream neural network, described in following sections.

### 1. Neural Function Approximator

In the disclosed embodiments, the favorable molecular conformations are predicted using an energy potential model.

In some embodiments, a neural network-based potential function approximator is used. The basic rationale is to train a smooth function approximation. This is a strong regularization term which makes the model prone to overfit and suitable for gradient based optimization.

The function approximator tries to learn a polynomial-like smooth potential function for any input embedding. The function approximator consists of three layers:
1. The first layer feeds the input embedding through a conventional fully connected layer to allow a linear transformation on the input:

X=Act(W₀X₀+b₀) (Eq. 2)

where in Act(x) is the activation function, X₀ is the input embedding, W and b are layer weights. A smooth activation function is used:

Swish(x)=x·sigmoid(x).
2. The second layer first transforms the input to logarithm scale, applying a scaling term and then uses an exponential function to transform the output back. In this way the scaling term corresponds to the exponential factor of the polynomial:

yᵢ=exp(w₁ᵢ·log(1+Xᵢ)) (Eq. 3)
3. The final layer applies a linear transformation of the polynomial output:

z=W₂y +b₂ (Eq. 4)

In some embodiments, symmetrical function approximation is used. Most smooth potential functions are symmetrical of the input atoms. For example, for the van der Walls (vdW) potential function of an unbonded atom pair i and j, the potential function vdW (i, j) is symmetrical, i.e., vdW (i, j)=vdW (j, i). However, for most neural networks, the input embeddings are ordered vectors, which violates the requirement of symmetry. To solve this problem, the set reduction function can be applied to those input groups whose order are irrelevant.

Specifically, let X=x₁, x₂, . . . , xₙ be the set of input embeddings whose ordering is irrelevant. The embeddings are first fed through a fully connected layer to determine the importance of each embedding:

t=Wx+b (Eq. 5)

The importance weights are normalized via the Softmax function:

$${t}_{i}^{′} = \frac{{e}^{{t}_{i}}}{{Σ}_{j}^{n} {e}^{{t}_{j}}} \qquad ( Eq . 6 )$$

Finally, all the embeddings are mixed according to calculated importance:

Reduce(X)=Reduce({X₁, X₂, . . . , Xₙ})=Σᵢⁿt′ⱼXᵢ (Eq. 7)

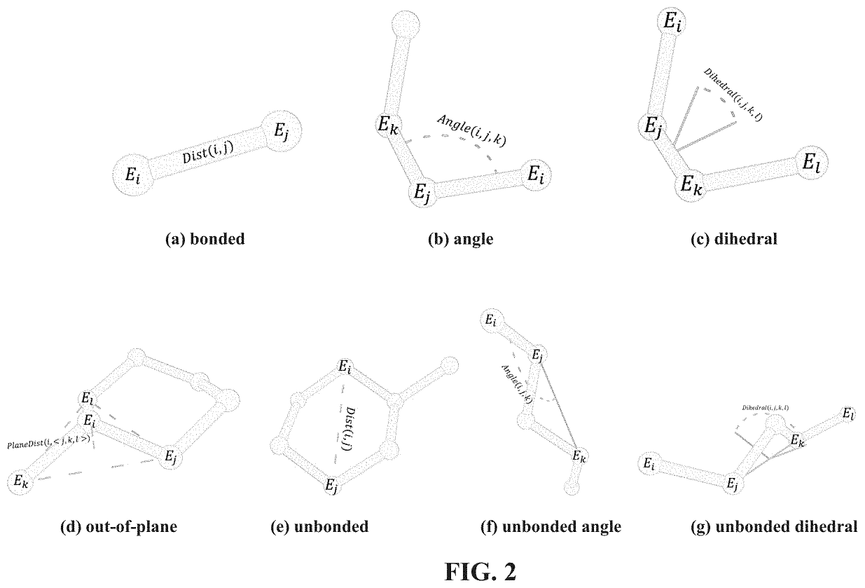

Theoretically, the above described function approximator model can be applied to any n-ary energy potential. FIG. 2 shows exemplary potentials used in the disclosed function approximator model, consistent with the disclosed embodiments. As shown in FIG. 2, the potentials may include:
Bonded potential: this measures the distance between a pair of bonded atoms. The bonded potential has the term for any bonded atoms i—j:

P_{b}(i,j)=F_{b}(Reduce(Eᵢ, Eⱼ), Dist(i,j), I_{b}(i,j)) (Eq. 8)

wherein Eᵢ and Eⱼ are the atom embeddings of the two atoms, Dist(i, j) is the Euclidean distance between the two atoms, I_{b}(i, j) is the ideal bond length of the two atoms, and F_{b} is the trained neural function approximator for the bond potential.
Angle potential: this measures the bond tension between pair of bonds. The angle potential has the term for any bonded atoms i—j—k:

Pₐ(i, j, k)=Fₐ(Eⱼ, Reduce(EᵢEₖ), Angle(i, j, k), Iₐ(i, j, k)) (Eq. 9)

wherein Eᵢ, Eⱼ and Eₖ are the atom embeddings of the three atoms, Angle(i, j, k) is the planar angle between the three atoms, Iₐ(i, j, k) is the ideal bond angle of the three atoms, and Fₐ is the trained neural function approximator for angle potential.
Dihedral potential: this measures the dihedral angle tensions between two planes. The dihedral potential has the term for any bonded atoms i—j—k—l:

P_{d}(i,j,k,l)=F_{d}(Reduce(Eᵢ, Eⱼ, Eₖ, Eₗ, Eₗ, Eₖ, Eⱼ, Eᵢ), Dihedral(i,j, k, l), I_{d}(i, j, k, l)) (Eq. 10)

wherein Eᵢ, Eⱼ, Eₖ and Eₗ are the atom embeddings of the four atoms, Dihedral(i, j, k, l) is the dihedral between the two planes <Eᵢ, Eⱼ, Eₖ> and <Eⱼ, Eₖ, Eₗ>, I_{d}(i, j, k, l) is the ideal dihedral angle of the four atoms, and F_{d} is the train neural function approximator for dihedral potential.
Out-of-plane potential: this measures the tension of planar atoms. The out-of-plane potential has the term for atoms j, k, l bonded to a central atom i:

Pₒₒₚ(i, j, k, l)=Fₒₒₚ(Eᵢ, Reduce(Eⱼ, Eₖ, Eₗ), PlaneDist(i, <j, k, l>)) (Eq. 11)

wherein Eᵢ, Eₖ and Eₗ are the atom embeddings of the four atoms, and PlaneDist(i, <j, k, l>) is the distance of the central atom i to the plane <j, k, l>. This term is added to atoms which is supposed to have planar bonds, such as sp or sp² hybridized carbons.
Unbonded pairwise potential: this measure distance between pair of atoms without a bond. This is similar to the bonded pairwise potential except for unbonded atoms. In other force fields this term might be divided into van Der Walls and electrostatic forces and parameterized separately. The unbonded pairwise potential has the term for any unbonded atoms pair i and j:

P_{ub}(i, j)=F_{ub}(Reduce(Eᵢ, Eⱼ), Dist(i, j)) (Eq. 12)

where Eᵢ and Eⱼ are the atom embeddings of the two atoms and Dist(i, j) is the Euclidean distance between the two unbonded atoms.
Unbonded angle potential and unbonded dihedral potential: these terms are added to model the anisotropic electron distributions of polar atoms. Both terms are similar to angle and dihedral potentials, except the unbonded angle Pᵤₐ is used for atoms i, j, k wherein i—j is bonded but k is not bonded to i, j:

Pᵤₐ(i, j, k)=Fᵤₐ(Eᵢ, Eⱼ, Eₖ, Angle(i, j, k)) (Eq. 13)

unbonded dihedral P_{ud} is used for atoms i, j, k, l where i—j and k—l are two bonded pairs, but there are no bonds in-between:

P_{ud}(i,j,k,l)=F_{ud}(Reduce(Eᵢ, Eⱼ, Eₖ, Eₗ, Eₗ, Eₖ, Eⱼ, Eᵢ), Dihedral(i,j,k,l)) (Eq. 14)

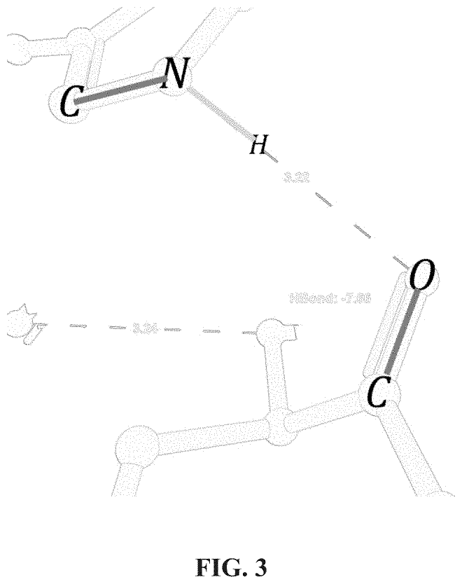

A typical situation wherein these two last terms are important is the hydrogen bond, which is very important in modeling inter-molecule binding interactions. As shown in FIG. 3, when hydrogen is not explicitly given, the hydrogen bond could only be deduced from the unbonded dihedrals, such as Dihedral(C—N—O—C). The anisotropy could be seen in the electron distribution of the oxygen atom in the carboxyl group. Thus, suppose there is an explicit H atom in the setting, then Angle(H—O—C) being input enables the ability of the system to describe the polarizability of atoms, and the formation criteria of a hydrogen bond.

The total potential function of a given molecule m is the sum of all extracted potential functions of the molecule:

$$\begin{matrix}P ( m ; θ ) = {\sum }_{( i , j ) ∈ B o n d s ( m )} {P}_{b} ( i , j ) + {\sum }_{( i , j , k ) ∈ A n g l e s ( m )} {P}_{a} ( i , j , k ) \\ + {\sum }_{( i , j , k , l ) ∈ D i hedral s ( m )} {P}_{d} ( i , j , k , l ) + {\sum }_{( i , j , k , l ) ∈ P l a n e s ( m )} {P}_{o o p} ( i , j , k , l ) \\ + {\sum }_{( i , j ) ∈ U nbondeds ( m )} {P}_{u b} ( i , j ) \\ + {\sum }_{( i , j , k ) ∈ U nbondedAngles ( m )} {P}_{u a} ( i , j , k ) \\ + {\sum }_{( i , j , k , l ) ∈ U nbondedDihedrals ( m )} {P}_{u d} ( i , j , k , l )\end{matrix} \qquad ( Eq . 15 )$$

wherein θ is the set of parameters of the function approximators. They are tuned during the training process and become fixed after the training is done.

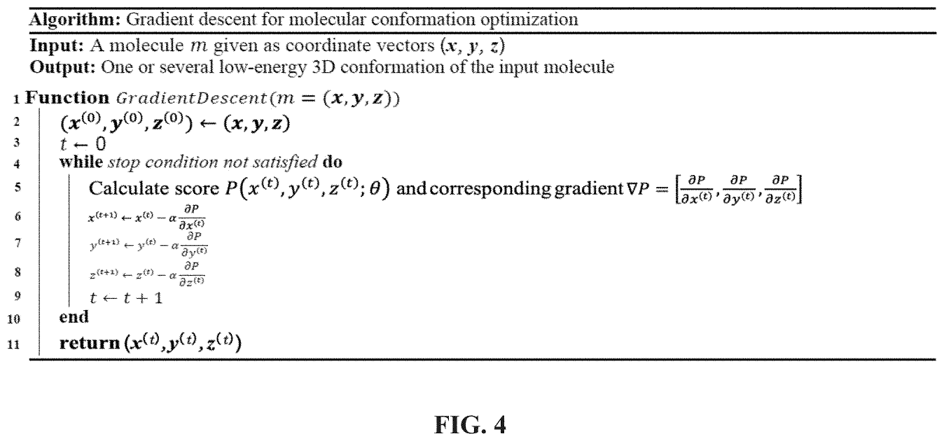

Given any molecule, such potential function can be defined. The information used to calculate the function is based on two parts of the molecule: one is the inherent graph structure of the molecule, such as elements of atoms, bonds, bond angles, and dihedrals. These are fixed regardless of the conformations of the molecule; the other one is the conformation of the molecule, which can be defined as positions of atoms in the molecule. Hence, P(m; θ)=P(x, y, z; θ) can be used, where (xᵢ, yᵢ, zᵢ) is the three-dimensional (3D) coordinates of atom i. A natural application of the potential function is to directly optimize the atom positions using gradient descent, similar to most Molecular Dynamics methods wherein a Newtonian energy and force model is applied. It is an iterative process as described in the algorithm shown in FIG. 4.

Notice that to make the gradient descent method possible, the function approximator becomes essential: It transfer the conventionally noncontinuous, non-smooth neural network models to a smooth, differentiable function.

Consistent with the disclosed embodiment, negative sampling can be used to train the potential model. The training process of the potential function P (m; θ) involves determining the parameters θ. It is relatively easy to obtain a large set of ground truth conformations, e.g., from crystal structures of ligands and proteins. Negative samples can be easily generated, by distorting existing conformations. However, most of such randomly generated distortions will lead to trivial negative samples, such as those with clashing (overlapping) atoms. Such examples contribute little value to model training.

To resolve this problem, different strategies can be used to sample negative examples. Such samples, by design, are intended to be at some good local minimums of the conformation space. The strategies used for the model training are described below:
Self-iterative training: this is a general way to make the potential approximators converge to a point where true ground truth molecules has the best potential. For any given molecule m, gradient descent algorithm can be used to optimize the conformation and get molecule m′. If the position deviation |m′−m| is larger than a predefined threshold, it means the model converges to a wrong local minimum. m′ is then added to the negative training examples.
Distorted side chain conformations: a rotamer library may be used to sample different rotamers of side chain conformations. For example, the rotamer library may be the library described in Ke Liu, Xiangyan Sun, Jun Ma, Zhenyu Zhou, Qilin Dong, Shengwen Peng, Junqiu Wu, Suocheng Tan, Gunter Blobel, and Jie Fan. 2017. Prediction of amino acid side chain conformation using a deep neural network. arXiv preprint arXiv:1707.08381 (2017), which is incorporated in the present disclosure by reference. The rotamers far from the ground truth position are added to the negative training examples. Moreover, gradient descent can be applied to those rotamers. And the results which are still far from the ground truth conformation can be added to the negative examples.
Distorted backbone conformations: the backbone atoms are distorted randomly and/or with backbone libraries. Gradient-descent-optimized examples are also added.
Docked conformations: First, a docking algorithm is used to dock ligands to protein pockets. Then, those docked conformations having a large difference from the crystal structure are added to the negative training examples.

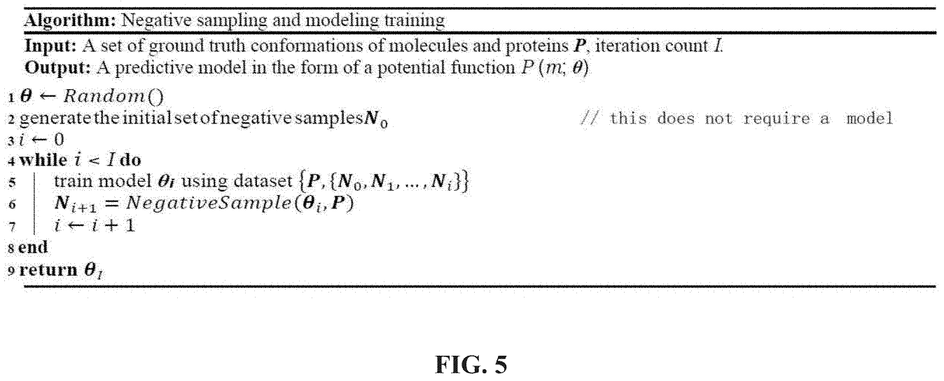

This creates a self-dependency (and a genetic iteration or evolution) of the model. To make this process possible, the model is trained in a bootstrapping setting. Formally, the algorithm is shown in FIG. 5.

There are two main classes of loss functions used in the training of potential function approximators:
Ranking loss: for each pair of molecule conformations (mₐ, m_{b}) wherein it is known (from ground truth) that conformation ma is more energetically stable than m_{b}, our goal is to ensure that our potential function has the relation P(mₐ; θ)<P (m_{b}; θ). Hence the loss function is defined as ReLU(P(m_{b}; θ)−P(mₐ; θ)).
Gradient loss: the potential function needs to converge, at least locally, to conformations with good confidence in their stability, such as conformations in crystalline structures. One way to achieve this is to make the gradient of the potential function at the targeted conformation approach zero. Hence the loss function is defined as a squared loss over the gradient of the targeted atom positions, i.e.

$$L = {( \frac{\partial P ( m ; θ )}{\partial m} )}^{2} = {( \frac{\partial P ( x , y , z ; θ )}{\partial x} + \frac{\partial P ( x , y , z ; θ )}{\partial y} + \frac{\partial P ( x , y , z ; θ )}{\partial z} )}^{2} \qquad ( Eq . 16 )$$

The disclosed potential model has various applications. In some embodiments, the disclosed potential model can be used to perform molecular conformation optimization. Once the potential models are defined, any molecule conformation can be directly optimized. As the functional approximator is designed to be smoothly differentiable. This process resembles molecular dynamics simulations.

This gradient descent scheme is efficient in finding local minimums of conformational energy. However, as in Molecular Dynamics simulation, the problem is not convex and a large number of such local minimums exists. It is hard to use gradient descent to cross high-energy barriers between distant conformations. For other tasks like sidechain conformation prediction, merely getting a set of independent local minima of sidechains are insufficient for the task. An iterative sampling-then-optimization strategy is then used as a general idea to circumvent this issue.

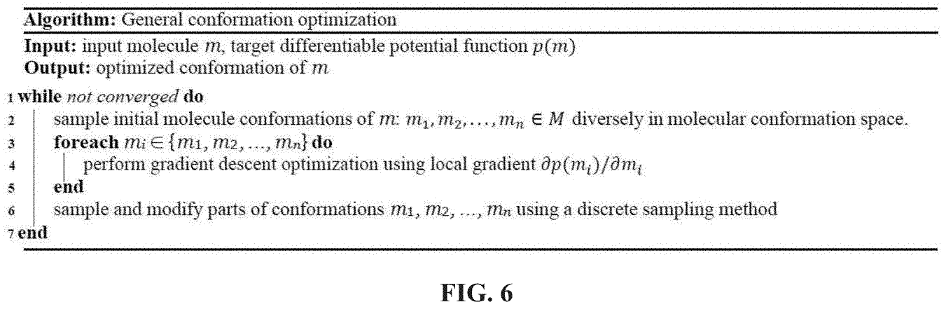

For any input molecule m and differentiable potential function p(m), the molecule is optimized using a general algorithm shown in FIG. 6.

Since the potential functions term p(m) is additive in terms of atom embeddings, it can be summed by either the whole molecule or some parts of a molecule. This makes the sampling algorithm much more flexible. In practice, the disclosed methods may start with sampling only part of a molecule and then extend the sampled part, as integrated in algorithms in the following sections.

In some embodiments, the disclosed potential model can be used to perform sidechain conformation prediction. The side chain conformation problem seeks to predict side chains of all amino acids given their fixed backbone conformations. This is a good testbed for molecular dynamics models in protein context. We examine this problem in the setting of leave-one-out prediction. That is to predict the side chain of a single amino acid with the environment fixed.

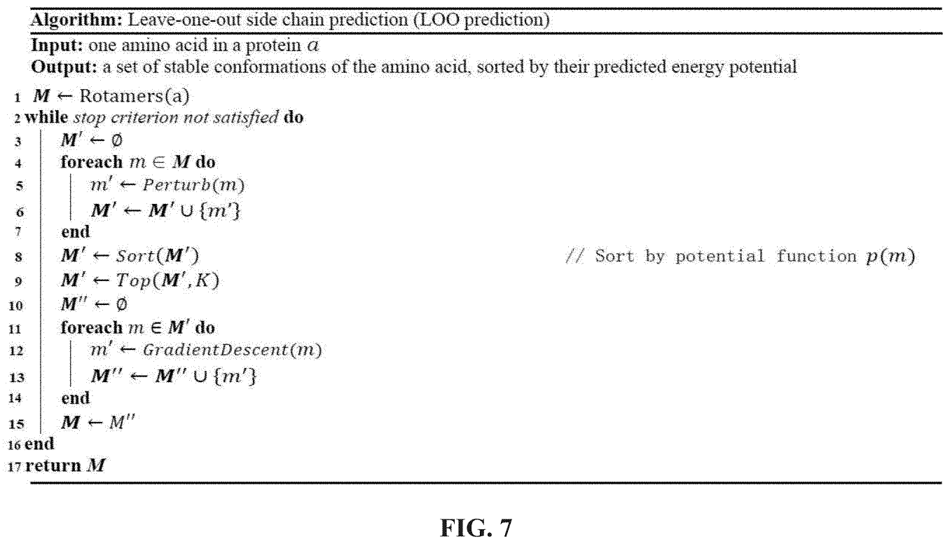

To effectively sample side chain conformations, we first build a backbone-independent rotamer library of side chain conformations, by which we reduce the leave-one-out prediction problem into two stages. Details of the backbone-independent rotamer library are described in the aforementioned reference: Ke Liu, Xiangyan Sun, Jun Ma, Zhenyu Zhou, Qilin Dong, Shengwen Peng, Junqiu Wu, Suocheng Tan, Gunter Blobel, and Jie Fan. 2017. Prediction of amino acid side chain conformation using a deep neural network. arXiv preprint arXiv: 1707.08381 (2017). The first stage is to test all existing rotamers of the side chain of an amino acid; the second stage is then to fine tune the best rotamers in the first stage, as shown by the algorithm shown in FIG. 7.

In the algorithm shown in FIG. 7, The rotamer library in line 1 is a small diverse set of potential conformations of each amino acid, generated from the training dataset as described in. The perturbation step in line 5 randomly perturbates the dihedral angles of the amino side chain in small steps. This is used to cross the barrier of many different dihedral configurations.

In some embodiments, the disclosed potential model can be used to perform ligand conformation generation. The ligand conformation generation problem seeks to generate correct 3D conformations of a given ligand structure depiction. It is usually used as a preparation step for downstream applications such as docking and property prediction. We solve the problem by taking the advantage of potential functions as different parts of the molecule can be independently sampled and having their potential score summed.

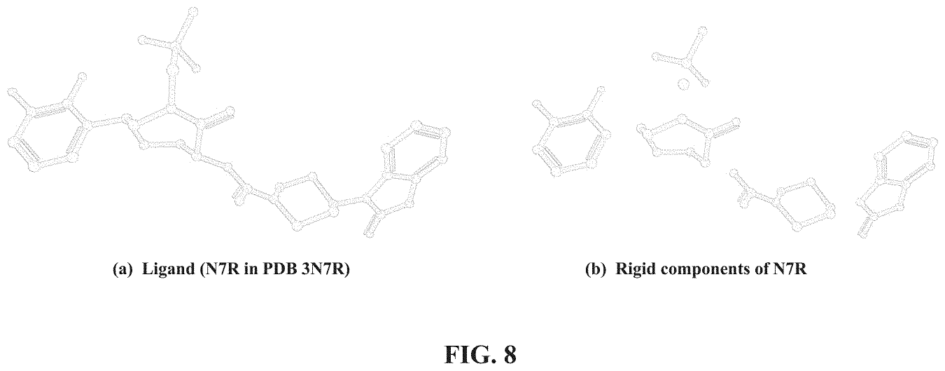

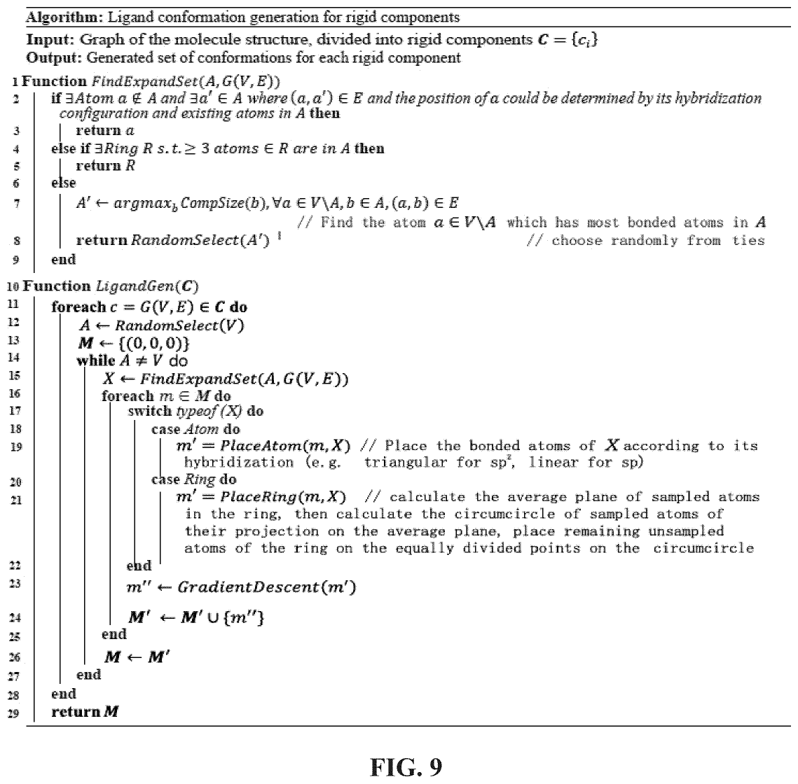

First, the input compound is divided into rigid components connected by rotatable bonds, as illustrated in FIG. 8. Each component is independently generated by repetitively sample atom conformations near to currently sampled atoms, starting from an empty conformation set. We maintain a set of partial conformations M which has the same set of sampled atoms. We repetitively decide the next atoms to sample and extend the partial conformations until the partial set equals the full set of the atoms of the input molecule. The formal procedure is described in the algorithm shown in FIG. 9.

After the conformations are generated for each rigid component, a clash avoiding sampling algorithm combined with gradient descent is used to connect the rigid components and sample good dihedral angle configuration of the rotatable bonds.

In some embodiments, the disclosed potential model can be used to perform ligand docking. Molecular docking refers to the problem of computing correct conformation of a ligand into a specified protein position (called a pocket). We use the anchor-and-grow method for molecular docking. The input ligand is divided into rigid components connected by rotatable bonds (FIG. 8). Then we repetitively place rigid components into the docked conformations, connecting it with existing atoms by rotating the dihedral of the connecting bond. During the docking process, only positions of parts of the final molecule is determined for each candidate conformation. The sampling algorithm repetitively extends docked components one by one to obtain the final docking result.

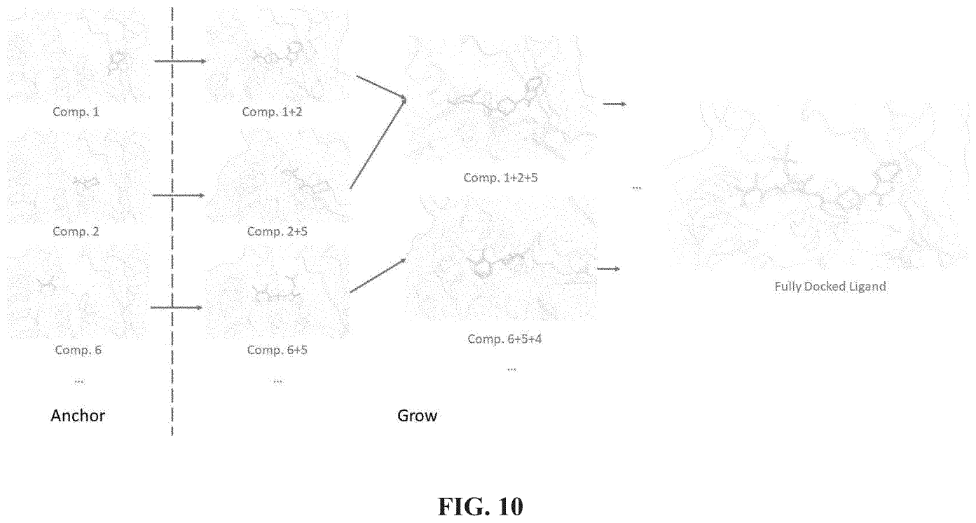

The docking algorithm is divided into the anchor and grow phases. In the anchor phase, the algorithm finds the best docking positions of each of the ligand's rigid components, resembling a simple rigid docking algorithm. In the grow phase, the algorithm extends existing partial docking conformations with other rigid components. It optimizes dihedral angles in this process. Two compatible conformations may also be merged. The grow phase is organized in a beam search setting. This process is illustrated in FIG. 10.

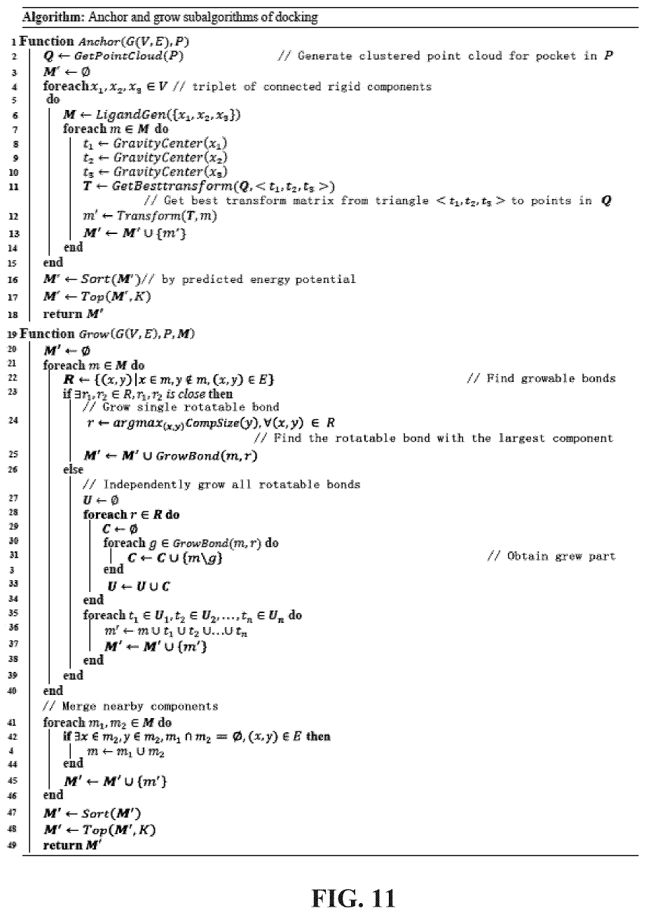

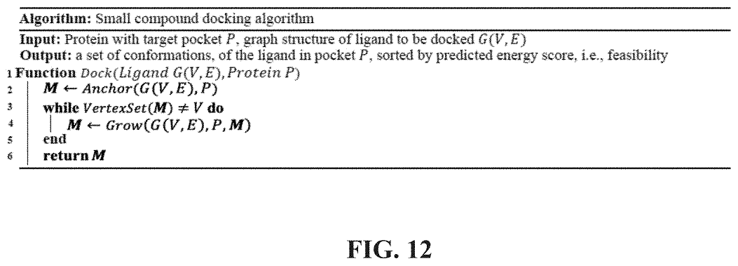

The algorithm corresponding to the process in FIG. 10 is shown in FIG. 11. Moreover, FIG. 12 shows an exemplary small compound docking algorithm. The input to the dock algorithm is the protein and the graph structure of input ligand G(V, E), where V contains one vertex for each of the rigid components and two components (x, [symbol] ) ∈ E iff rigid components x and [symbol] is connected by a single rotatable bond.

To show the actual performance of the disclosed model in real-life scenarios, several experiments have been set up, with comparisons with state-of-the-art methods in those particular fields.

For example, the disclosed model is used to test side chain prediction. In the exissitng SCWRL4 program, three kinds of description of the accuracy of the model were given. Here the correctness is defined as having a difference of angle less than 40°, and the numbering of x angles starts from the closest dihedral angle to the backbone:
1. The absolute probability that Xᵢ, Xᵢ₋₁, . . . , X₁ is correct, for all residues and for each type of amino acid.
2. The root mean squared deviation (RMSD) of the side chain residues. The RMSD values are calculated by adding up the RMSD value of each residue and are then averaged. The RMSD of a single residue is calculated using the formula:

$$RMSD ( v , w ) = \sqrt{\frac{1}{n} {\sum }_{i = 1}^{n} {‖ {v}_{i} - {w}_{i} ‖}^{2}} \qquad ( Eq . 17 )$$

We have tested our model and SCWRL4 on SCWRL4's dataset comprising 379 PDB files. The result is shown in Table 1 below. The results show clear superiority of our method over SCWRL4 by having both lower RMSD and higher x accuracies on all amino acids.

TABLE 1
RMSD and x value comparison between SCWRL4 and our method for LOO prediction on 379 proteins

| Amino | | RMSD | | x₁ | | x₂ | | x₃ | | x₄ | | x₅ | |
|---|---|---|---|---|---|---|---|---|---|---|---|---|---|
| Acid | Count | Scwrl4 | Ours | Scwrl4 | Ours | Scwrl4 | Ours | Scwrl4 | Ours | Scwrl4 | Ours | Scwrl4 | Ours |
| ALA | 5888 | 0.044 | 0.038 | | | | | | | | | | |
| ARG | 3719 | 1.866 | 1.365 | 83.0 | 88.9 | 71.6 | 78.6 | 48.4 | 61.4 | 38.0 | 52.1 | 38.0 | 52.1 |
| ASN | 2948 | 0.622 | 0.487 | 88.5 | 91.5 | 79.8 | 86.1 | | | | | | |
| ASP | 4142 | 0.621 | 0.458 | 88.0 | 92.4 | 79.6 | 86.0 | | | | | | |
| CYS | 1052 | 0.308 | 0.192 | 92.6 | 96.4 | | | | | | | | |
| GLN | 2590 | 1.157 | 0.919 | 82.8 | 87.6 | 67.2 | 74.9 | 52.0 | 66.5 | | | | |
| GLU | 4751 | 1.137 | 0.993 | 78.4 | 83.3 | 66.4 | 71.6 | 49.7 | 60.2 | | | | |
| GLY | 5547 | 0.000 | 0.000 | | | | | | | | | | |
| HIS | 1562 | 0.744 | 0.478 | 92.9 | 95.5 | 85.0 | 92.2 | | | | | | |
| ILE | 4058 | 0.352 | 0.281 | 96.6 | 97.6 | 86.7 | 90.0 | | | | | | |
| LEU | 6729 | 0.447 | 0.342 | 94.7 | 96.7 | 88.9 | 91.0 | | | | | | |
| LYS | 3995 | 1.414 | 1.216 | 80.1 | 87.0 | 68.6 | 76.0 | 54.1 | 59.3 | 35.4 | 38.4 | | |
| MET | 1430 | 0.970 | 0.651 | 86.6 | 92 8 | 75.9 | 87.3 | 59.9 | 71.7 | | | | |
| PHE | 2800 | 0.576 | 0.304 | 97.4 | 99.3 | 95.9 | 98.9 | | | | | | |
| PRO | 3319 | 0.210 | 0.173 | 87.0 | 91.6 | 83.3 | 88.0 | | | | | | |
| SER | 4210 | 0.539 | 0.416 | 72.8 | 80.4 | | | | | | | | |
| THR | 3920 | 0.316 | 0.247 | 90.8 | 93.8 | | | | | | | | |
| TRP | 1008 | 1.001 | 0.432 | 94.7 | 98.7 | 87.7 | 96.0 | | | | | | |
| TYR | 2416 | 0.687 | 0.379 | 97.2 | 99.2 | 95.4 | 98.4 | | | | | | |
| VAL | 5138 | 0.261 | 0.211 | 93.4 | 95.3 | | | | | | | | |

As another example, the disclosed model has been used to test compound docking. The testing set is our filtered protein data bank (PDB) database for the purpose of reliable benchmarking: PDB Docking Set v2 (PDSv2).

The criterion is listed as follow:
1. The PDB structure is determined by X-ray diffraction with a resolution <2.5 Å.
2. The ligand should belong to a protein with type “protein”.
3. The ligand should be connected (in a graph theory sense), NOT being a solvent molecule, and not bonded to any atoms that is not part of itself.
4. The ligand should have >5 non-hydrogen atoms in the ligand, no more than 8 atoms in its largest simple ring, and ≤10 rotatable bonds.
5. There should be no external metal atoms within 3.0 Å from the ligand.
6. There should be no Hetero atoms with the same Residue name with 5.0 Å.
7. There should be no external atoms within 3.0 Å from the metal atom in the ligand.
8. The ratio of complete amino acids, i.e., amino acids without missing atoms, should be larger than 90%.

A total of 1441 high quality protein ligand complex structures are selected this way for benchmarking.

We do our testing against several other widely-accepted docking algorithms, including UCSF Dock (William J Allen, Trent E Balius, Sudipto Mukherjee, Scott R Brozell, Demetri T Moustakas, P Therese Lang, David A Case, Irwin D Kuntz, and Robert C Rizzo. 2015. DOCK 6: Impact of new features and current docking performance. Journal of computational chemistry 36, 15 (2015), 1132-1156), AutoDock Vina (Oleg Trott and Arthur J Olson. 2010. AutoDock Vina: improving the speed and accuracy of docking with a new scoring function, efficient optimization, and multithreading. Journal of computational chemistry 31, 2 (2010), 455-461), and Rosetta (Samuel DeLuca, Karen Khar, and Jens Meiler. 2015. Fully flexible docking of medium sized ligand libraries with RosettaLigand. PLOS one 10, 7 (2015), e0132508). The results are shown in Table 2 below. RMSD is calculated between positions of each docked atom vᵢ and its corresponding ground truth position wᵢ. Special treatment is done to handle symmetrical positions so that flipping a benzene ring will result in zero RMSD. We also considered a modified RMSD metric called the shape RMSD, which for each docked atom vᵢ, the position wᵢ corresponds to the nearest atom in the ground truth conformation, which may not be the same atom in the molecule. A low shape RMSD indicates the algorithm is able to fit the molecule into the shape of the pocket.

TABLE 2
RMSD and shape RMSD comparison between our methods and other methods.
@k indicates the best evaluation score of the first k of the predicted
conformations, sorted by the algorithm's internal ranking score

| | ShapeRMSD@1 | ShapeRMSD@5 | RMSD@1 | RMSD@5 | Success Count |
|---|---|---|---|---|---|
| Ours | 0.858651293 | 0.637538778 | 1.769472363 | 1.032891272 | 1441 |
| UCSF-FLX-FLEXIBLE | 1.352609299 | 0.890155312 | 2.634079219 | 1.484691792 | 1431 |
| UCSF-FLX-RIGID | 1.831002016 | 1.250156037 | 3.814842201 | 2.497495059 | 1433 |
| AutoDock Vina | 1.354831147 | 0.895898547 | 2.770003983 | 1.586554709 | 1433 |
| Rosetta | 1.291723616 | 0.923275125 | 2.755971552 | 1.728418176 | 1418 |

It is clear from the results that our machine-learned model performs much better than all previous state-of-the-art methods.

As described above, molecular force field construction has many different applications in drug discovery and molecular modeling. The present disclosure provides a novel method of using neural networks to train a potential function. It combines the benefits of traditional handcrafted potential functions as being smoothly differentiable, with the benefits of being fully automatically-trained from large crystal structure databases. We tested the trained potential function and showed it has superior performance over existing molecular force fields, without the need of any manual parameter tuning.

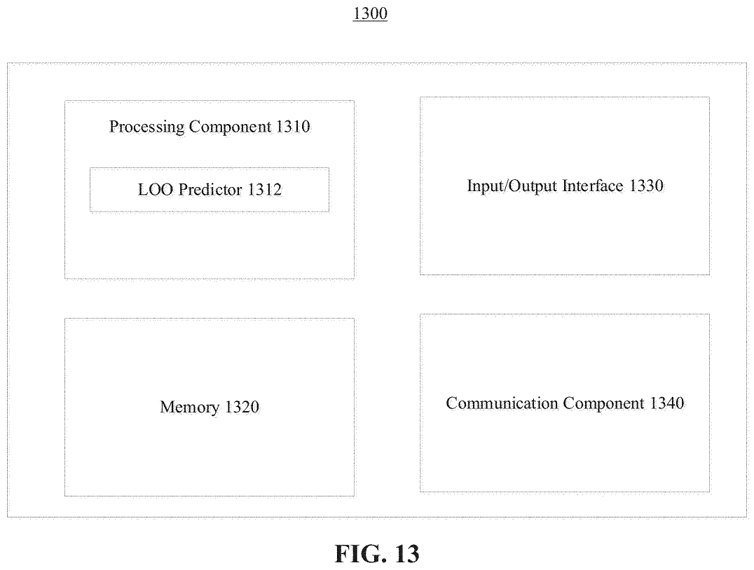

FIG. 13 is a block diagram of a device 1300 for performing the disclosed methods, according to an exemplary embodiment. For example, device 1300 may be a desktop, a laptop, a server, a server cluster consisting of a plurality of servers, a cloud computing service center, etc. Referring to FIG. 13, device 1300 may include one or more of a processing component 1310, a memory 1320, an input/out (I/O) interface 1330, and a communication component 1340.

Processing component 1310 may control overall operations of device 1300. For example, processing component 1310 may include one or more processors that execute instructions to perform all or part of the steps in the foregoing described methods. In particular, processing component 1310 may include a LOO predictor 1312 configured to use the disclosed machine-learning methods to generate the LOO models, and to execute the LOO models to perform the disclosed modular modeling methods. Further, processing component 1310 may include one or more modules (not shown) which facilitate the interaction between processing component 1310 and other components. For instance, processing component 1310 may include an I/O module to facilitate the interaction between I/O interface and processing component 1310.

Processing component 1310 may include one or more application specific integrated circuits (ASICs), digital signal processors (DSPs), digital signal processing devices (DSPDs), programmable logic devices (PLDs), field programmable gate arrays (FPGAs), controllers, micro-controllers, microprocessors, or other electronic components, for performing all or part of the steps in the above-described methods.

Memory 1320 is configured to store various types of data and/or instructions to support the operation of device 1300. Memory 1320 may include a non-transitory computer-readable storage medium including instructions for applications or methods operated on device 2200, executable by the one or more processors of device 1300. For example, the non-transitory computer-readable storage medium may be a read-only memory (ROM), a random access memory (RAM), a CD-ROM, a magnetic tape, a memory chip (or integrated circuit), a hard disc, a floppy disc, an optical data storage device, or the like.

I/O interface 1330 provides an interface between the processing component 1310 and peripheral interface modules, such as input and output devices of device 1300. I/O interface 1330 may employ communication protocols/methods such as audio, analog, digital, serial bus, universal serial bus (USB), infrared, PS/2, BNC, coaxial, RF antennas, Bluetooth, etc. For example, I/O interface 1330 may receive user commands from the input devices and send the user commands to processing command 1310 for further processing.

Communication component 1340 is configured to facilitate communication, wired or wirelessly, between device 1300 and other devices, such as devices connected to the Internet. Communication component 1340 can access a wireless network based on one or more communication standards, such as Wi-Fi, LTE, 2G, 3G, 4G, 5G, etc. In some embodiments, communication component 1340 may be implemented based on a radio frequency identification (RFID) technology, an infrared data association (IrDA) technology, an ultra-wideband (UWB) technology, a Bluetooth (BT) technology, or other technologies. For example, communication component 1340 may access the molecular databases (e.g., Protein Data Bank) via the Internet and/or send the molecular modeling results to a user.

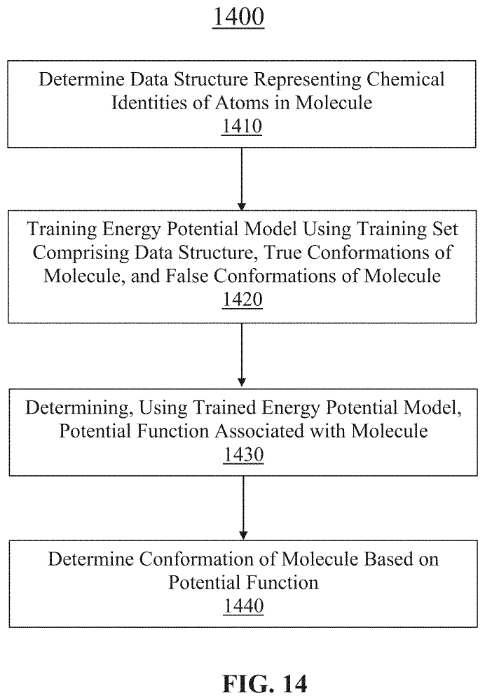

FIG. 14 illustrates a flow chart of a neural network based method 1400 for predicting molecular conformation, according to some embodiments of the present disclosure. Method 1400 can be performed by a computing device (e.g., device 1300 of FIG. 13) or any software or hardware components thereof. In some embodiments, method 1400 can be implemented by a computer program product, embodied in a computer-readable medium, including computer-executable instructions, such as program code, executed by computers (e.g., device 1300 of FIG. 13). Referring to FIG. 14, method 1400 may include the following steps 1410-1440.

In step 1410, the computing device may determine a data structure representing chemical identities of atoms in a molecule. Consistent with the disclosed embodiments, the molecule involved in the determination may be an amino acid side chain or a ligand docked in another molecule, such as a protein. The data structure may be embeddings of atom types.

In some embodiments, input to the neural network may include a graphic representation of the molecule. The computing device may extract features of the atoms from an input file of the neural network. The features to be extracted include, but are not limited to: an element type of a first atom in the molecule; an electrostatic charge of the first atom; a van der Wells radius or a covalent radius of the first atom; information indicating whether the first atom is part of a ring; information indicating whether the first atom is part of an aromatic ring; information indicating whether the first atom forms a single, double, triple, or aromatic bond; information indicating whether the first atom and a second atom of the molecule are in a same ring, etc. The computing device may compute the embeddings of atom types based on the extracted features.

In step 1420, the computing device may train an energy potential model using a training set including the data structure, true conformations of the molecule, and false conformations of the molecule.

in step 1430, the computing device may determine, using the trained energy potential model, a potential function associated with the molecule. In some embodiments, the computing device may convert the data structure into a neural network based smooth potential function. The smooth potential function may include potential terms representing one or more of: a bonded potential; an angle potential; a dihedral potential; an out of plane potential; an unbonded pairwise potential; an unbonded angle potential; an unbonded dihedral potential, etc.

In step 1440, the computing device may determine a conformation of the molecule based on potential function. For example, the computing device may find conformations corresponding to local minimums of the potential function. The found conformations are those conformation likely existing in reality. They can be used in downstream analysis, such as protein conformation prediction, drug design, etc.

In some embodiments, a non-transitory computer-readable storage medium including instructions is also provided, and the instructions may be executed by a device (such as the disclosed encoder and decoder), for performing the above-described methods. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM or any other flash memory, NVRAM, a cache, a register, any other memory chip or cartridge, and networked versions of the same. The device may include one or more processors (CPUs), an input/output interface, a network interface, and/or a memory.

As used herein, unless specifically stated otherwise, the term “or” encompasses all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above described embodiments can be implemented by hardware, or software (program codes), or a combination of hardware and software. If implemented by software, it may be stored in the above-described computer-readable media. The software, when executed by the processor can perform the disclosed methods. The computing units and other functional units described in the present disclosure can be implemented by hardware, or software, or a combination of hardware and software. One of ordinary skill in the art will also understand that multiple ones of the above described modules/units may be combined as one module/unit, and each of the above described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only, with a true scope and spirit of the invention being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

In the drawings and specification, there have been disclosed exemplary embodiments. However, many variations and modifications can be made to these embodiments. Accordingly, although specific terms are employed, they are used in a generic and descriptive sense only and not for purposes of limitation.

What is claimed is:

1. A computer-implemented method for predicting molecular conformation, the method comprising:
determining a data structure representing chemical identities of atoms in a molecule;
training an energy potential model using a training set comprising the data structure, true conformations of the molecule, and false conformations of the molecule;
determining, using the trained energy potential model, a potential function associated with the molecule; or
determining a conformation of the molecule based on potential function.

2. The method according to claim 1, wherein determining the data structure representing the chemical identities of the atoms in the molecule comprises:
training a neural network to determine embeddings of atom types associated with the atoms.

3. The method according to claim 2, wherein input to the neural network comprises a graphic representation of the molecule.

4. The method according to claim 2, further comprising:
extracting features of the atoms from an input file of the neural network, wherein the features includes one or more of:
an element type of a first atom in the molecule,
an electrostatic charge of the first atom,
a van der Wells radius or a covalent radius of the first atom,
information indicating whether the first atom is part of a ring,
information indicating whether the first atom is part of an aromatic ring,
information indicating whether the first atom forms a single, double, triple, or aromatic bond, or
information indicating whether the first atom and a second atom of the molecule are in a same ring.

5. The method according to claim 1, wherein determining the potential function associated with the molecule comprises:
converting the data structure into a neural network based smooth potential function.

6. The method according to claim 5, wherein the smooth potential function comprises potential terms representing one or more of:
a bonded potential,
an angle potential,
a dihedral potential,
an out of plane potential,
an unbonded pairwise potential,
an unbonded angle potential, or
an unbonded dihedral potential.

7. The method according to claim 1, wherein the molecule is an amino acid side chain or a ligand docked in another molecule.

8. An apparatus for predicting molecular conformation, the apparatus comprising:
at least one memory for storing instructions; and
at least one processor configured to execute the instructions to cause the apparatus to perform:
determining a data structure representing chemical identities of atoms in a molecule;
training an energy potential model using a training set comprising the data structure, true conformations of the molecule, and false conformations of the molecule;
determining, using the trained energy potential model, a potential function associated with the molecule; or
determining a conformation of the molecule based on potential function.

9. The apparatus according to claim 8, wherein in determining the data structure representing the chemical identities of the atoms in the molecule, the at least one processor is configured to execute the instructions to cause the apparatus to perform:
training a neural network to determine embeddings of atom types associated with the atoms.

10. The apparatus according to claim 9, wherein input to the neural network comprises a graphic representation of the molecule.

11. The apparatus according to claim 9, wherein the at least one processor is configured to execute the instructions to cause the apparatus to perform:
extracting features of the atoms from an input file of the neural network, wherein the features includes one or more of:
an element type of a first atom in the molecule,
an electrostatic charge of the first atom,
a van der Wells radius or a covalent radius of the first atom,
information indicating whether the first atom is part of a ring,
information indicating whether the first atom is part of an aromatic ring,
information indicating whether the first atom forms a single, double, triple, or aromatic bond, or
information indicating whether the first atom and a second atom of the molecule are in a same ring.

12. The apparatus according to claim 8, wherein in determining the potential function associated with the molecule, the at least one processor is configured to execute the instructions to cause the apparatus to perform:
converting the data structure into a neural network based smooth potential function.

13. The apparatus according to claim 12, wherein the smooth potential function comprises potential terms representing one or more of:
a bonded potential,
an angle potential,
a dihedral potential,
an out of plane potential,
an unbonded pairwise potential,
an unbonded angle potential, and
an unbonded dihedral potential.

14. The apparatus according to claim 8, wherein the molecule is an amino acid side chain or a ligand docked in another molecule.

15. A non-transitory computer readable storage medium storing a set of instructions that are executable by one or more processing devices to cause an apparatus to perform a method comprising:
determining a data structure representing chemical identities of atoms in a molecule;
training an energy potential model using a training set comprising the data structure, true conformations of the molecule, and false conformations of the molecule;
determining, using the trained energy potential model, a potential function associated with the molecule; and
determining a conformation of the molecule based on potential function.

16. The non-transitory computer readable storage medium according to claim 15, wherein determining the data structure representing the chemical identities of the atoms in the molecule comprises:
training a neural network to determine embeddings of atom types associated with the atoms.

17. The non-transitory computer readable storage medium according to claim 16, wherein input to the neural network comprises a graphic representation of the molecule.

18. The non-transitory computer readable storage medium according to claim 16, further comprising:
extracting features of the atoms from an input file of the neural network, wherein the features includes one or more of:
an element type of a first atom in the molecule,
an electrostatic charge of the first atom,
a van der Wells radius or a covalent radius of the first atom,
information indicating whether the first atom is part of a ring,
information indicating whether the first atom is part of an aromatic ring,
information indicating whether the first atom forms a single, double, triple, or aromatic bond, and
information indicating whether the first atom and a second atom of the molecule are in a same ring.

19. The non-transitory computer readable storage medium according to claim 15, wherein determining the potential function associated with the molecule comprises:
converting the data structure into a neural network based smooth potential function.

20. The non-transitory computer readable storage medium according to claim 19, wherein the smooth potential function comprises potential terms representing one or more of:
a bonded potential,
an angle potential,
a dihedral potential,
an out of plane potential,
an unbonded pairwise potential,
an unbonded pairwise angle potential, or
an unbonded pairwise dihedral potential.
